(19) United States
(12) Patent Application Publication
(10) Pub. No.: US 2021/0059593 A1
(43) Pub. Date: Mar. 4, 2021

(54) METHOD AND SYSTEM FOR DETECTING AND QUANTIFYING CHEMICAL SPECIES IN A SAMPLE USING ANIMAL DETECTION

(71) Applicant: Chevron U.S.A. Inc., San Ramon, CA (US)

(72) Inventor: Daniel Concannon SEGAL, Oakland, CA (US)

(21) Appl. No.: 16/555,830

(22) Filed: Aug. 29, 2019

(51) Int. Cl. A61B 5/00; A01K 15/02; G01N 1/28

(52) U.S. Cl. CPC A61B 5/4011; G01N 2001/002; G01N 1/28; A01K 15/02

(57) ABSTRACT

Disclosed are systems and methods for detecting and quantifying chemicals in a sample using an animal having been trained to detect the chemical. A lower detection limit at which the animal can detect the chemical is determine. The animal is enclosed in a chamber, and air is introduced to the chamber in a series of tests having a different ratio of filtered air not having contacted the sample to air from the sample at increasing concentrations of the chemical until the animal detects the chemical. Then the concentration of the chemical in the sample is approximated by dividing the lower detection limit at which the non-human animal detects the chemical species by a ratio of a volume of air in contact with the sample to a total volume of air entering the chamber at which the non-human animal detects the chemical.

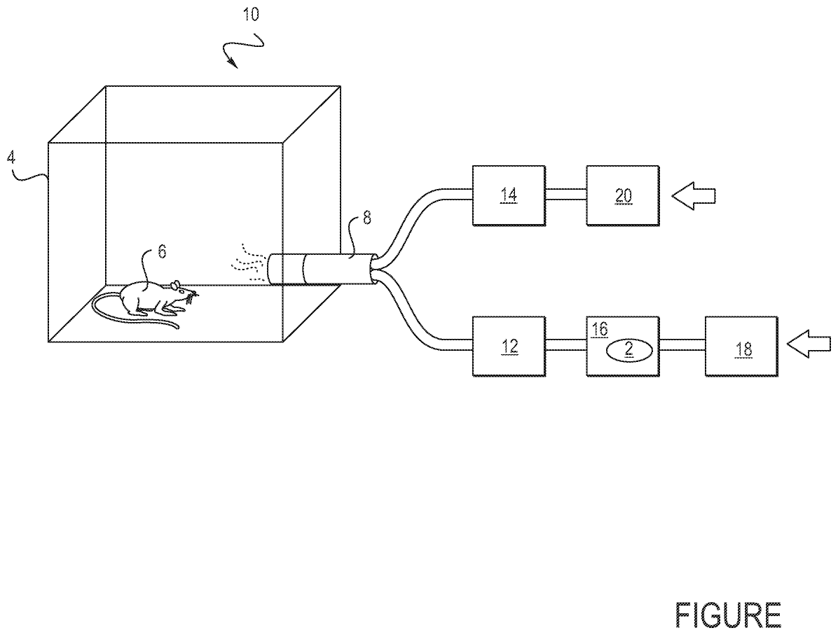

### FIELD

The present disclosure relates generally to the field of quantifying chemical species in a sample using animal sensing.

### BACKGROUND

Current laboratory methods for quantifying chemicals in samples (including solid, liquid, gas and mixed-media) have a number of drawbacks. Traditional laboratory equipment is often not portable, slow, expensive and prone to error. Sensing animals are routinely used by law enforcement to detect contraband because of their mobility and speed. Dogs have also been trained to detect the oil buried beneath sand or snow during oil spill cleanup operations. However, these applications using sensing animals are not quantitative, i.e., they only provide an indication of presence or absence of a substance.

It would be desirable to have a method for generating a quantification of chemicals in a way that is mobile, quick, accurate and cost-effective.

### SUMMARY

In general, in one aspect, the disclosure relates to a method for detecting and quantifying a chemical species in a sample. The method includes determining a lower detection limit of a non-human animal having been trained to detect a chemical species at which the non-human animal can detect the chemical species. The non-human animal is enclosed in a chamber having an air inlet connected to the chamber for introducing air to the chamber. The air inlet can receive air containing air from the sample and air not having contacted the sample in varying relative amounts. Air is introduced to the chamber through the air inlet in a series of separate tests wherein each test has a different ratio of air not having contacted the sample to air from the sample at increasing concentrations of the chemical species presented to the non-human animal in the chamber until the non-human animal detects the chemical species. Then the concentration of the chemical species in the sample is approximated by dividing the lower detection limit at which the non-human animal detects the chemical species by a ratio of a volume of air in contact with the sample to a total volume of air entering the chamber at which the non-human animal detects the chemical.

In another aspect, the disclosure can generally relate to a system for detecting and quantifying a chemical species in a sample. The system includes a chamber for containing the non-human animal trained to detect the chemical species, an air inlet connected to the chamber for introducing air to the chamber wherein the air inlet can receive air containing air from the sample and air not having contacted the sample in varying relative amounts, a first variable pump upstream of the air inlet for supplying the air from the sample to the air inlet, and a second variable pump for supplying the air not having contacted the sample to the air inlet. The first and second variable pumps can be controlled to vary the ratio of the air not having contacted the sample to the air from the sample fed to the chamber.

### BRIEF DESCRIPTION OF THE DRAWINGS

These and other objects, features and advantages of the present invention will become better understood with reference to the following description, appended claims and accompanying drawings. The drawings are not considered limiting of the scope of the appended claims. Reference numerals designate like or corresponding, but not necessarily identical, elements. The drawings illustrate only example embodiments. The elements and features shown in the drawings are not necessarily to scale, emphasis instead being placed upon clearly illustrating the principles of the example embodiments. Additionally, certain dimensions or positionings may be exaggerated to help visually convey such principles.

The FIGURE shows a schematic diagram of a system in which example embodiments can be applied.

### DETAILED DESCRIPTION

Referring to the FIGURE, in one embodiment, a system 10 for detecting and quantifying a chemical species in a sample 2 is shown. The sample 2 can be in solid, liquid, gas or mixed-media form, e.g., soil, and can be contained in a container 16. A variable pump 12 (having a variable speed) is placed in fluid communication with the container 16 to supply air from the sample 2 to a chamber 4 for containing a non-human animal 6 having been trained to detect the chemical species of interest. The chamber 4 is substantially airtight and can be made from any suitable solid material. Nonlimiting examples of suitable animals 6 include dogs and rats, which have an extremely sensitive sense of smell and may detect concentrations well below regulatory limits for environmental contaminants, e.g., total petroleum hydrocarbons (TPH) in soil samples. An air inlet 8 is connected to the chamber 4 for introducing the air to the chamber 4. The air inlet 8 can receive air containing air from the sample 2 as well as air not having contacted the sample 2. The air inlet 8 can receive air from both sources in varying relative amounts by varying the speeds on both the variable pump 12 and a second variable pump 14 for supplying the air not having contacted the sample 2 to the air inlet 8. The variable pumps 12 and 14 can be controlled to vary the ratio of the air not having contacted the sample 2 to the air from the sample 2.

The concentration of the chemical species delivered to the chamber 4 by the air inlet 8 can be approximated by dividing a lower detection limit (LDL) at which the non-human animal 6 can detect the chemical species by the ratio of a volume of the air in contact with the sample 2 to a total volume of air entering the chamber 4 at which the non-human animal 6 detects the chemical.

In one embodiment, an air filter 18 can be placed in fluid communication with and upstream of the container 16 for receiving ambient air and filtering the ambient air prior to contact of the ambient air with the sample 2 in the container 16. Likewise, in one embodiment, an air filter 20 can be placed in fluid communication with and upstream of the second variable pump 14 for receiving ambient air and filtering the ambient air prior to the ambient air being pumped into the air inlet 8.

In one embodiment, a method or protocol for detecting and quantifying a chemical species of interest in a sample 2 includes the following steps. First, a lower detection limit (LDL) of the non-human animal 6, having already been trained to detect the chemical species, at which the non-human animal 6 can detect the chemical species is determined. This LDL is the average lowest concentration in the animal 6 responds to. With the non-human animal 6 enclosed in the chamber 4, air is introduced to the chamber 4 through the air inlet 8 in a series of separate tests. In each test, a different ratio of air not having contacted the sample 2 to air from the sample 2, i.e., air having contacted the sample 2, at increasing concentrations of the chemical species is presented to the non-human animal 6 in the chamber 4 until the non-human animal 6 detects the chemical species. The concentration of the chemical species in the sample 2 can then be approximated by dividing the LDL at which the non-human animal 6 was found to be able to detect the chemical species by a dilution ratio ratio (DR) of a volume of air in contact with the sample 2 (“soil sample air” pumped by variable pump 12) to a total volume of air (“total air” including soil sample air and “filtered air” pumped by variable pump 14) entering the chamber 4 at which the non-human animal 6 detects the chemical, i.e.:

Concentration=LDL/DR, where

DR=soil sample air/total air

To illustrate, the table below illustrates an example method to determine TPH concentration.

TABLE

| | | Filtered | Soil | TPH Concentration Calculation | |
|---|---|---|---|---|---|
| Test | | Air | Sample Air | Animal Response | No Animal Response |
| 1 | Blank | 100% | 0% | Test Failed | Next Test |
| 2 | TPH | 0% | 100% | [TPH] > LDL | [TPH] < LDL |
| | presence | | | Next Test | End Test |
| 3 | DR = 0.1 | 90% | 10% | [TPH] > LDL/DR | LDL/DR > [TPH] > LDL |
| | | | | Next Test | End Test |
| 4 | DR = 0.2 | 80% | 20% | [TPH] > LDL/DR | LDL/DR > [TPH] > |
| | | | | | previous LDL/DR |
| | | | | Next Test | End Test |
| 5 | DR = 0.3 | 70% | 30% | [TPH] > LDL/DR | LDL/DR > [TPH] > |
| | | | | | previous LDL/DR |
| | | | | Next Test | End Test |
| 6 | DR = 0.4 | 60% | 40% | [TPH] > LDL/DR | LDL/DR > [TPH] > |
| | | | | | previous LDL/DR |
| | | | | Next Test | End Test |
| 7 | DR = 0.5 | 50% | 50% | [TPH] > LDL/DR | LDL/DR > [TPH] > |
| | | | | | previous LDL/DR |
| | | | | Next Test | End Test |
| 8 | DR = 0.6 | 40% | 60% | [TPH] > LDL/DR | LDL/DR > [TPH] > |
| | | | | | previous LDL/DR |
| | | | | Next Test | End Test |
| 9 | DR = 0.7 | 30% | 70% | [TPH] > LDL/DR | LDL/DR > [TPH] > |
| | | | | | previous LDL/DR |
| | | | | Next Test | End Test |
| 10 | DR = 0.8 | 20% | 80% | [TPH] > LDL/DR | LDL/DR > [TPH] > |
| | | | | | previous LDL/DR |
| | | | | Next Test | End Test |
| 11 | DR = 0.9 | 10% | 90% | [TPH] > LDL/DR | LDL/DR > [TPH] > |
| | | | | | previous LDL/DR |
| | | | | End Test | End Test |

The disclosed system and method can be used for a variety of applications, including assisting in determining the amount of and mapping the location of TPH in soil at a site. The disclosed system and method can also be used to quantify chemical constituents and process facilities.

It should be noted that only the components relevant to the disclosure are shown in the figures, and that many other components normally part of a chemical species detection system are not shown for simplicity.

For the purposes of this specification and appended claims, unless otherwise indicated, all numbers expressing quantities, percentages or proportions, and other numerical values used in the specification and claims are to be understood as being modified in all instances by the term “about.” Accordingly, unless indicated to the contrary, the numerical parameters set forth in the following specification and attached claims are approximations that can vary depending upon the desired properties sought to be obtained by the present invention. It is noted that, as used in this specification and the appended claims, the singular forms “a,” “an,” and “the,” include plural references unless expressly and unequivocally limited to one referent.

Unless otherwise specified, the recitation of a genus of elements, materials or other components, from which an individual component or mixture of components can be selected, is intended to include all possible sub-generic combinations of the listed components and mixtures thereof. Also, “comprise,” “include” and its variants, are intended to be non-limiting, such that recitation of items in a list is not to the exclusion of other like items that may also be useful in the materials, compositions, methods and systems of this invention.

What is claimed is:

1. A method for detecting and quantifying a chemical species in a sample comprising the chemical species, comprising:
a. determining a lower detection limit of a non-human animal trained to detect the chemical species at which the non-human animal can detect the chemical species;
b. enclosing the non-human animal in a chamber having an air inlet connected to the chamber for introducing air to the chamber wherein the air inlet can receive air containing air from the sample and air not having contacted the sample in varying relative amounts;
c. introducing air to the chamber through the air inlet in a series of separate tests wherein each test has a different ratio of air not having contacted the sample to air from the sample at increasing concentrations of the chemical species presented to the non-human animal in the chamber until the non-human animal detects the chemical species; and
d. approximating the concentration of the chemical species in the sample by dividing the lower detection limit at which the non-human animal can detect the chemical species by a ratio of a volume of air in contact with the sample to a total volume of air entering the chamber at which the non-human animal detects the chemical.

2. The method of claim 1, further comprising pumping the air from the sample by a first variable pump upstream of the air inlet from the container to the air inlet, and pumping the air not having contacted the sample by a second variable pump from an ambient air source to the air inlet.

3. The method of claim 2 further comprising filtering the air from the ambient air source prior to pumping the air not having contacted the sample.

4. The method of claim 1, further comprising filtering an air stream at a location upstream of the sample prior to contacting the sample.

5. A system for detecting and quantifying a chemical species in a sample comprising the chemical species, comprising:
a. a chamber for containing a non-human animal trained to detect the chemical species;
b. an air inlet connected to the chamber for introducing air to the chamber wherein the air inlet can receive air containing air from the sample and air not having contacted the sample in varying relative amounts;
c. a first variable pump upstream of the air inlet for supplying the air from the sample to the air inlet; and
d. a second variable pump for supplying the air not having contacted the sample to the air inlet;
wherein the first and second variable pumps can be controlled to vary a ratio of the air not having contacted the sample to the air from the sample and thereby approximate a concentration of the chemical species delivered to the chamber by the air inlet by dividing a lower detection limit at which the non-human animal can detect the chemical species by the ratio of a volume of the air in contact with the sample to a total volume of air entering the chamber at which the non-human animal detects the chemical.

6. The system of claim 5 wherein the sample can be located in a container in fluid communication with and upstream of the first variable pump such that air from the sample is pumped by the first variable pump from the container to the air inlet.

7. The system of claim 5 further comprising an air filter located in fluid communication with and upstream of the container for receiving ambient air and filtering the ambient air prior to contact of the ambient air with the sample in the container.

8. The system of claim 5 further comprising an air filter located in fluid communication with and upstream of the second variable pump for receiving ambient air and filtering the ambient air prior to the ambient air being pumped into the air inlet.
